(19) United States
(12) Patent Application Publication
(10) Pub. No.: US 2019/0153072 A1
(43) Pub. Date: May 23, 2019

(54) METHOD FOR PRODUCING ANTIBODY FRAGMENT

(71) Applicant: KANEKA CORPORATION, Osaka (JP)

(72) Inventors: Dai Murata, Hyogo (JP); Shinichi Yoshida, Hyogo (JP)

(73) Assignee: KANEKA CORPORATION, Osaka (JP)

(21) Appl. No.: 16/259,174

(22) Filed: Jan. 28, 2019

Related U.S. Application Data
(63) Continuation of application No. PCT/JP2017/025226, filed on Jul. 11, 2017.

(30) Foreign Application Priority Data
Jul. 28, 2016 (JP) 2016-148820

(51) Int. Cl. C07K 16/06; B01D 15/38

(52) U.S. Cl. CPC C07K 16/065; C07K 2317/522; C07K 2317/55; B01D 15/3809

(57) ABSTRACT

A method for producing an antibody fragment includes preparing a liquid sample that includes the antibody fragment and does not include an Fc fragment, adsorbing the antibody fragment on an affinity separation matrix by contacting the liquid sample with the affinity separation matrix, removing impurities from the affinity separation matrix by washing the affinity separation matrix, and separating the antibody fragment from the affinity separation matrix. The antibody fragment includes a CH1 region and does not include an Fc region. The affinity separation matrix includes a water-insoluble carrier and a ligand immobilized on the water-insoluble carrier. The ligand is Protein G, a Protein G domain, a Protein G variant or a Protein G domain variant.

### TECHNICAL FIELD

One or more embodiments of the present invention relate to a method for producing an antibody fragment which has a higher purity and which contains a CH1 region but does not contain an Fc region.

### BACKGROUND

As one of important functions of a protein, an ability to specifically bind to a specific molecule is exemplified. The function plays an important role in an immunoreaction and signal transduction in a living body. A technology utilizing the function for purifying a useful substance has been actively developed. As one example of proteins which are actually utilized industrially, for example, a Protein A affinity separation matrix has been used for purifying an antibody drug with high purity at one time from a culture of an animal cell (Non-patent documents 1 and 2). Hereinafter, Protein A is abbreviated as “SpA” in some cases.

An antibody drug which has been developed is mainly a monoclonal antibody, and a monoclonal antibody has been produced on a large scale by using recombinant cell cultivation technology. A “monoclonal antibody” means an antibody obtained from a clone derived from a single antibody-producing cell. Most of antibody drugs which are presently launched are classified into an immunoglobulin G (IgG) subclass in terms of a molecular structure. Accordingly, in an initial purification step of an antibody drug production, an SpA affinity separation matrix containing SpA, which specifically binds to an Fc region of IgG, is utilized.

An antibody fragment, has a molecular structure obtained by fragmenting an immunoglobulin, and various antibody fragment drugs have been clinically developed (Non-patent Document 3). Among an antibody fragment, an SpA affinity separation matrix cannot be used for purifying an antibody fragment which does not contain an Fc region. Accordingly, an affinity separation matrix capable of adsorbing an antibody fragment which does not contain an Fc region of IgG is highly required industrially in terms of a platform process for purifying an antibody drug.

A plurality of proteins which can bind to a region except for an Fc region of IgG have been already known (Non-patent Document 4). For example, as an affinity separation matrix having Protein L as a ligand, Capto L™, KappaSelect and LambdaFabSelect are known. Protein L binds to a Fab region of IgG. Hereinafter, Protein L is abbreviated as “SpL” in some cases. KappaSelect and LambdaFabSelect have a camel antibody as a ligand. The above-described affinity separation matrixes recognize only either of κ light chain or λ light chain.

The protein referred to as Protein G binds to IgG. Protein G was found from Streptococcus sp. classified in Group G. Hereinafter, Protein G is abbreviated as “SpG” in some cases. There is an SpG affinity separation matrix product (product name: “Protein G Sepharose 4 Fast Flow” manufactured by GE Healthcare, Patent Document 1) prepared by immobilizing SpG as a ligand. SpG strongly binds to an Fc region of IgG and it is also known that SpG weakly binds to a Fab region (Non-patent. Documents 4 and 5). It is reported in Patent document 2 that F(ab′)₂ obtained by cleaving IgG with protease is purified by an SpG affinity separation matrix on the basis this property.

### Patent Document

Patent Document 1: JP S63-503032 T

Patent Document 2: JP H4-49300 A

### Non-Patent Document

Non-patent Document 1: Hober S., et al., Chromatogr. B, 2007, vol. 848, pp. 40-47

Non-patent. Document 2: Shukla A. A., et al., Trends Biotechnol., 2010, vol. 28, pp. 253-261

Non-patent Document 3: Nelson A. N., et. al., Nat. Biotechnol., 2009, vol. 27, pp. 331-337

Non-patent Document 4: Bouvet P. J., Int. J. Immunopharmac., 1994, vol. 16, pp. 419-424

Non-patent. Document 5: Derrick J. P., Nature, 1992, vol. 359, pp. 752-754

Non-patent Document 6: Andre F., et al., frontiers in IMMUNOLOGY, 2013, vol. 217, pp. 1-20

Non-patent Document 7: KOBAYASHI Kazuo, et al., Seibutsu-kougaku Kaishi, 2008, vol. 86, pp. 390-392

As described above, IgG is generally purified by using an affinity separation matrix containing SpA, and a ligand and an elution condition have been sufficiently studied. On the one hand, with respect to an antibody fragment, though there are a plurality of affinity separation matrixes used for purifying an antibody fragment, sufficient study has not been made.

For example, it is known to produce an antibody fragment by cleaving IgG with a protease to fragment IgG and purifying a target antibody fragment as described above. This method, however, is inefficient, since it is needed to purify a target antibody fragment from a mixture of a Fab fragment and an Fc fragment, a mixture of F(ab′)₂ fragment and a further cleaved Fc fragment, or the like. Accordingly, it has been considered to selectively produce a target antibody fragment by a genetic engineering technology. Even in this method by a genetic engineering technology, a misfolded compound and unwanted component derived from an antibody are excessively produced all together in some cases in addition to a target antibody fragment (Non-patent Documents 6 and 7).

It can be thought that, for example, a Fab fragment is efficiently produced by selectively producing the Fab fragment and using an affinity separation matrix having SpL, which is a protein binding to a light chain, as a ligand with the above-described latter method. On the one hand, the present inventors found by an experiment that when a Fab fragment is produced by a genetic engineering technology, an impurity such as a light chain monomer and a light chain dimer is produced and immixed into the target Fab fragment, since SpL also adsorbs such an impurity. As described above, it is difficult to purify a target antibody fragment, since an impurity having a similar physical property to the target antibody fragment is produced.

### SUMMARY

One or more embodiments of the present invention provide a method for efficiently producing a target antibody fragment having high purity by separating an impurity which has a similar physical property to the target antibody fragment and of which separation is difficult.

The inventors intensively studied and found that SpG has a specific affinity for an antibody fragment which contains a CH1 region and which does not contain an Fc region but has a low affinity for an impurity which contains a part of the target antibody fragment as a constituent element, such as a light chain monomer and a light chain dimer. Accordingly, the present inventors completed one or more embodiments of the present invention by finding that when an antibody fragment which contains a CH1 region and which does not contain an Fc region is purified from a sample which does not contain an Fc fragment, an impurity of which separation is difficult by a conventional method can be effectively separated and the target antibody fragment can be recovered with higher purity by using an affinity separation matrix having SpG as a ligand.

Hereinafter, one or more embodiments of the present invention are described.
[1] A method for producing an antibody fragment,

wherein the antibody fragment comprises a CH1 region and does not comprise an Fc region,

comprising the steps of:

preparing a liquid sample, wherein the liquid sample comprises the antibody fragment and does not comprise an Fc fragment,

contacting the liquid sample with an affinity separation matrix in order to adsorb the antibody fragment on the affinity separation matrix, wherein Protein G, a Protein G domain, a Protein G variant or a Protein G domain variant is immobilized as a ligand on a water-insoluble carrier in the affinity separation matrix,
washing the affinity separation matrix to remove an impurity, and

separating the antibody fragment from the affinity separation matrix.
[2] The method according to the above [1], wherein an association constant of the Protein G variant or the Protein G domain variant to the CH1 region is 10⁶ M⁻¹ or more.
[3] The method according to the above [2], wherein the Protein G variant or the Protein G domain variant has an amino acid sequence of SEQ ID NO: 5.
[4] The method according to any one of the above [1] to [3], wherein the antibody fragment comprises a light chain, and wherein the impurity is 1 or more selected from the group consisting of a light chain monomer, a light chain dimer and an antibody aggregate.
[5] The method according to any one of the above [1] to [4], wherein an amount of a washing liquid used for washing the affinity separation matrix is 3 column volume or more.
[6] The method according to any one of the above [1] to [5], wherein an eluate used for separating the antibody fragment from the affinity separation matrix is an aqueous solution of 1 or more acids selected from the group consisting of acetic acid, citric acid and glycine.
[7] The method according to the above [6], wherein a pH of the aqueous solution is 2.5 or more and 4.0 or less.

According to one or more embodiments of the present invention method, an impurity of which physical property is similar to a target antibody fragment, such as a misfolded compound, can be removed and the target antibody fragment can be purified with higher purity after a method for selectively producing the target antibody fragment more efficiently by using a genetic engineering technology in comparison with a method for fragmenting an antibody and further purifying the target antibody fragment. Thus, according to one or more embodiments of the present invention method, it becomes possible to reduce the load of examining the condition in the latter purification stage and it becomes easy to build a purification process, since an impurity of which physical property is similar to a target antibody fragment is not brought into the latter purification stage.

### BRIEF DESCRIPTION OF THE DRAWINGS

FIG. 1 is a chromatogram in the case where a CH1 region-containing Fab fragment in a culture supernatant of a yeast transfectant was purified by using a Protein G variant carrier prepared by immobilizing a Protein G variant into which mutations were introduced.

FIG. 2 is a chromatogram in the case where a CH1 region-containing Fab fragment in a culture supernatant of a yeast transfectant was purified by using a commercially available Protein G affinity separation matrix prepared by immobilizing a wild Protein G.

FIG. 3 is a chromatogram in the case where a CH1 region-containing Fab fragment in a culture supernatant of a yeast transfectant was purified by using a commercially available Protein L affinity separation matrix prepared by immobilizing Protein L.

FIG. 4 is the SDS-PAGE result of each fraction in FIG. 1 and FIG. 2 in a reductive condition and a non-reductive condition.

FIG. 5 is an expanded figure around 45 to 66 kDa of the SDS-PAGE gel of FIG. 4 in a non-reductive condition.

FIG. 6 is the SDS-PAGE result of each fraction in FIG. 3 in a reductive condition and a non-reductive condition.

### DETAILED DESCRIPTION OF THE EMBODIMENTS

In one or more embodiments of the present invention method, an antibody fragment which contains a CH1 region and which does not contain an Fc region is efficiently purified with high purity by using an affinity separation matrix on which Protein G, a Protein G domain, a Protein G variant or a Protein G domain variant is immobilized. Hereinafter, each step of one or more embodiments of the present invention method is described.

Step 1: Step of Preparing Crude Antibody Fragment Sample

In Step 1, a liquid sample which contains an antibody fragment containing a CH1 region and not containing an Fc region and which does not contain an Fc fragment is prepared. A method for preparing such a liquid sample is not particularly restricted as long as a target antibody fragment can be selectively produced and an impurity containing an Fc region is not produced as a by-product by the method. For example, the liquid sample can be prepared by a genetic engineering technology. The term “genetic engineering technology” in one or more embodiments of the present invention means that a gene encoding a target antibody fragment is introduced into a cell to obtain a transformant and the transformant is cultivated to selectively produce the target antibody fragment. In addition, the liquid sample can be also produced by a cell-free protein synthesis system using a gene encoding a target antibody fragment.

An “immunoglobulin (Ig)” is a glycoprotein produced by a B cell of a lymphocyte and has a function to recognize a specific molecule such as a protein to be bound. An immunoglobulin has not only a function to specifically bind to a specific molecule referred to as antigen but also a function to detoxify and remove an antigen-containing factor in cooperation with other biological molecule or cell. An immunoglobulin is generally referred to as an “antibody”, and the name is inspired by such functions.

All of immunoglobulins basically have the same molecular structure. The basic structure of an immunoglobulin is a Y-shaped four-chain structure. The four-chain structure is composed of two light chains and two heavy chains of polypeptide chains. A light chain (L chain) is classified into two types of λ chain and κ chain, and all of immunoglobulins have either of the chains. A heavy chain (H chain) is classified into five types of γ chain, μ chain, α chain, δ chain and ε chain, and an immunoglobulin is classified into isotypes depending on the kind of a heavy chain. An immunoglobulin G (IgG) is a monomer immunoglobulin, is composed of two γ chains and two light chains, and has two antigen-binding sites.

A lower half vertical part in the “Y” shape of an immunoglobulin is referred to as an “Fc region”, and an upper half “V” shaped part is referred to as a “Fab region”. An Fc region has an effector function to initiate a reaction after an antibody binds to an antigen, and a Fab region has a function to bind to an antigen. A Fab region of a heavy chain and an Fc region are bound to each other through a hinge part. Papain, which is a proteolytic enzyme and which is contained in papaya, decomposes a hinge part to cut into two Fab regions and one Fc region. The domain part close to the tip of the “Y” shape in a Fab region is referred to as a “variable region (V region)”, since there are various changes of the amino acid sequence in order to bind to various antigens. A variable region of a light chain is referred to as a “VL region”, and a variable region of a heavy chain is referred to as a “VH region”. A Fab region except for a V region and an Fc region are referred to as a “constant region (C region)”, since there is relatively less change. A constant region of a light chain is referred to as a “CL region”, and a constant region or a heavy chain is referred to as a “CH region”. A CH region is further classified into three regions of CH1 to CH3. A Fab region of a heavy chain is composed of a VH region and a CH1 region, and an Fc region of a heavy chain is composed of a CH2 region and a CH3 region. There a hinge part between a CH1 region and a CH2 region. More specifically, it is known that the binding of Protein G to a Fab region corresponds to the binding of IgG to a CH1 region (CH1γ) and a CL region (Non-patent Document 5), and the present inventors experimentally found that when a target antibody fragment containing a CH1 region is purified by Protein G, an impurity which does not contain a CH1 region and which contains a CL region can be removed.

When an antibody is cleaved by papain, two Fab fragments and one Fc fragment are obtained. When an antibody is cleaved by pepsin, one F(ab′)₂ and Fc fragments cut apart are obtained. Thus, it is not comparatively efficient to purify an antibody fragment which contains a CH1 region and which does not contain an Fc region from the above-described mixture. On the one hand, a genetic engineering technology and a cell-free protein synthesis system are comparatively efficient.

In one or more embodiments of the present invention, a liquid sample which contains an antibody fragment containing a CH1 region and not containing an Fc region and which does not an Fc fragment is prepared. The antibody fragment is not particularly restricted as long as the antibody fragment contains a CH1 region and does riot contain an Fc region and may be a Fab fragment prepared by fragmenting an immunoglobulin G to a Fab region only, a F(ab′)₂ fragment, a Fab₂ fragment, a Fab₃ fragment, a fragment covalently bound by a drug, and a complex prepared by fusing the fragments with a recombination protein.

In Step 1, a gene encoding a target antibody fragment may be prepared by an ordinary method. For example, a gene encoding a target antibody fragment is chemically synthesized and amplified by PCR, or DNA containing a gene encoding a target antibody fragment is used as a template and the gene encoding the target antibody fragment is amplified by PCR using primers capable of amplifying the gene. Since the antibody fragment to be produced in one or more embodiments of the present invention method contains a CH1 region and does not contain an Fc region, a gene encoding an Fc region is not used. In the “Fc region” in one or more embodiments of the present invention, a part of an Fc region, such as a CH2 region and a CH3 region, is also included.

Next, a gene encoding a target antibody fragment is inserted into a vector. A vector to be inserted by the gene is not particularly restricted as long as the vector is capable of autonomous replication in a host, and plasmid DNA and phage DNA can be used as the vector. In one or more embodiments, it is preferred that the vector contains a promoter which can function in a host. As the host, a fungus such as yeast; a bacterium such as Escherichia coli and Bacillus subtilis; an animal cell such as Chinese hamster ovary (CHO) cell, BHK cell, COS cell and human-derived cell; an insect cell can be used. A host into which the above-described gene is inserted is cultivated to produce the gene. After cultivation, the used host cell is removed by filtration, centrifugation or the like to obtain the liquid sample containing a target antibody fragment. Since a gene encoding an Fc region is not used in Step 1, an Fc fragment is not contained in the liquid sample.

In one or more embodiments, the pH of the liquid sample is preferably neutral as about 6 or more and about 8 or less. A solvent of the liquid sample may be water only or a buffer solution having pH of about 6 or more and about 8 or less and may contain a water-miscible organic solvent such as C₁₋₄ alcohol as long as a main component of the solvent is water.

Step 2: Adsorption Step

In Step 2, the liquid sample is contacted with an affinity separation matrix prepared by immobilizing Protein G, a Protein G domain, a Protein G variant or a Protein G domain variant on an insoluble carrier as a ligand in order to adsorb the antibody fragment on the affinity separation matrix.

The affinity separation matrix used in Step 2 is prepared by immobilizing Protein G, a Protein G domain, a Protein G variant or a Protein G domain variant on an insoluble carrier as a ligand. Hereinafter, the Protein G, Protein G domain, Protein G variant or Protein G domain variant is collectively referred to as “Protein G ligand” in some cases.

Protein G (SpG) is a protein derived from a cell wall of Streptococcus sp. classified in Group G. Protein G has an ability to bind to an immunoglobulin G (IgG) of most mammals, and it is known that SpG strongly binds to an Fc region of IgG. SpG also weakly binds to a Fab region of IgG, particularly a CH1 region and a CL region.

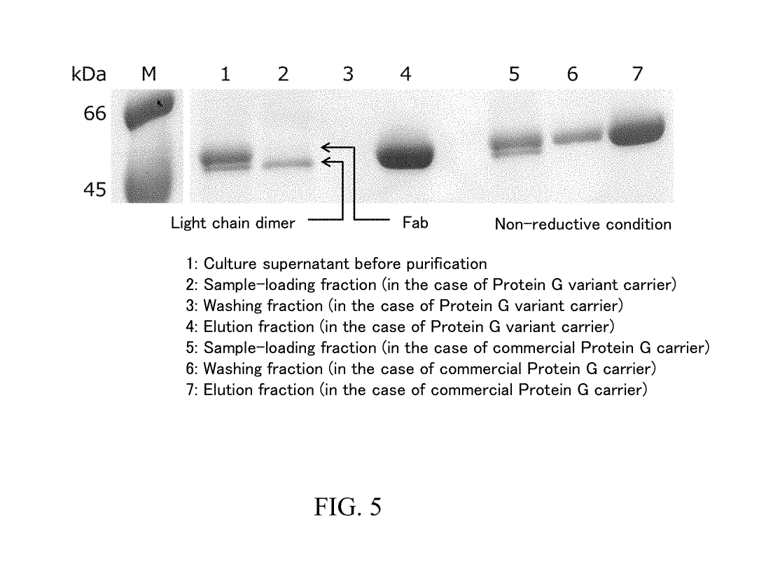

An SpG functional domain having an IgG-binding ability is referred to as “βdomain”, i.e. “SpG-β”. The domain is referred to as β (B) domain or C domain. (refer to Akerstrom et al., J. Biol. Chem., 1987, 28, p.13388-, FIG. 5), but is referred to as “β domain” in this disclosure in accordance with the definition described in Fahnestock et al. (Fahnestock et al., J. Bacteriol., 1986, 167, p.870-). The details of the amino acid sequence of SpG-β are different depending on the kind and strain of a bacterium from which the SpG-β is derived. As the typical amino acid sequences, with respect to two β domains (β1 and β2) derived from Group G Streptococcus sp. GX7809 strain, the amino acid sequence of β31 domain (SpG-β1) is shown as SEQ ID NO: 2 and the amino acid sequence of β2 domain (SpG-β2) is shown as SEQ ID NO: 2 Each β domain of SpG is collectively referred to as Protein G-β domain (SpG-β), since the amino acid sequences of each β domain of SpG have high sequence identity with each other.

The term “domain” means a unit of higher-order structure of a protein. For example, a domain is composed of from dozens to hundreds of amino acid residues, and means a protein unit which can sufficiently serve some kind of a physicochemical or biochemical function.

The term “variant” of a protein or peptide means a protein or peptide obtained by introducing at least one substitution, addition and/or deletion into an amino acid sequence of a wild protein or peptide. The affinity of the “variant” in one or more embodiments of the present invention for a CH1 region is at least maintained or improved in comparison with wild SpG or a domain thereof.

Protein G (SpG) is a protein which contains 2 or 3 IgG-binding domains in the form of tandem line. The Protein G ligand used as a ligand of the affinity separation matrix according to one or more embodiments of the present invention may be a multimer of 2 or more IgG-binding domains of wild SpG and/or SpG variant as monomers or single domains. In one or more embodiments, the number of the monomers or single domains is preferably 3 or more, more preferably 4 or more, and even more preferably 5 or more. In one or more embodiments, the upper limit of the number of connected domains may be, for example, 10, preferably 8, and more preferably 6. Such a multimer may be a homomultimer in which one kind of IgG-binding domains of wild SpG and/or SpG variant are connected, such as homodimer and homotrimer, or a heteromultimer in which two or more kinds of IgG-binding domains of wild SpG and/or SpG variant are connected, such as heterodimer and heterotrimer. As described above, 1 or more amino acids may be also added to the multimer composed of a plurality of domains. In one or more embodiments, the position to be added is preferably an N-terminal and a C-terminal. For example, Cys may be added to a C-terminal of 2 domain type of the IgG-binding domain of wild SpG and/or SpG variant.

In one or more embodiments, it is preferred that an association constant of an SpG variant or an SpG domain variant to a CH1 region is 10⁶ M⁻¹ or more. The association constant of the Protein G ligand as a ligand of the affinity separation matrix according to one or more embodiments of the present invention to a CH1 region can be evaluated by a biosensor such as Biacore system (manufactured by GE Healthcare Bioscience) utilizing surface plasmon resonance and Octet system (manufactured by Pall) utilizing biolayer interferometry, but the means is not restricted thereto. As an association constant parameter to evaluate an affinity for a CH1 region, for example, an association constant (K_{A}) and a dissociation constant (K_{D}) can be used (Nagata et al., “Real-Time Analysis Experimental Method for Interaction Between Biological Substances”, Springer-Verlag Tokyo, 1998, p. 41). A material used for evaluating an association constant of the Protein G ligand to a CH1 region is exemplified by a CH1 region peptide prepared by a genetic engineering technology; however, the CH1 region prepared by this method may not have a right structure. As another means, an association constant to 2 kinds of Fab having different antigen-recognition sites may be considered to be a quasi-association constant to a CH1 region. For example, 2 kinds of IgG of which amino acid sequences are known, of which antigen recognition sites are different from each other but of which CH1 region homology is high are respectively fragmented by an enzyme and purified to be Fab fragments to be used. If association constants to 2 kind of Fab fragments are nearly equal, the measured association constant can be considered to be a quasi-association constant to a CH1 region.

The upper limit of an association constant to a CH1 region is not particularly restricted. In one or more embodiments, the higher association constant is preferred, since an antibody fragment as a target molecule which contains a CH1 region and which does not contain an Fc region can be strongly adsorbed. On the one hand, when the association constant is excessively high, a low pH solution is needed for dissociating an adsorbed target antibody fragment; as a result, the target antibody fragment, may be damaged. Accordingly, in one or more embodiments, the above-described association constant is preferably, for example, 10¹¹ M⁻¹ or less.

In the experiment using Biacore system or Octet system, the order of the parameter may be largely changed depending on an experimental condition, analysis method and/or the kind of the original antibody fragment. As one of judgmental standards in such a case, it is exemplified whether a binding constant is larger or not when a peptide such as wild Protein G is evaluated an the same experimental condition and by the same analysis method. Wild Protein G is easily available as a commercially available research reagent manufactured by, for example, Life Technology.

As an SpG domain variant, the variant having the amino acid sequence of SEQ ID NO: 5 is exemplified. The SpG domain variant having the amino acid sequence of SEQ ID NO: 5 has an especially high affinity for a Fab fragment and can purify an antibody fragment containing a CH1 region and not containing an Fc region more efficiently.

A method for connecting monomer proteins used as a ligand of the affinity separation matrix according to one or more embodiments of the present invention is exemplified by a connecting method through one or more amino acid residues but is not restricted thereto. The number of the amino acid residue for connection is not particularly restricted and may be preferably 20 residues or less, and more preferably 15 residues or less. In one or more embodiments, it is preferred to use a sequence which connects β1 and β2 or β2 and β3 of wild SpG. From another point of view, it may be preferred that the amino acid residue for connection does not destabilize a three dimensional structure of the monomer protein.

The ligand of the affinity separation matrix according to one or more embodiments of the present invention may be a fusogenic peptide containing an IgG-binding domain of wild SpG and/or a SpG variant or a peptide multimer composed of the 2 or more domains connected each other as one constituent and other peptide having a different function. Such a fusogenic peptide is exemplified by a peptide fused with albumin or GST, i.e. glutathione S-transferase, but is not restricted to the examples. In addition, peptides fused with a nucleic acid such as DNA aptamer, a drug such as antibiotic or a polymer such as PEG, i.e. polyethylene glycol, are also included in the scope of the present invention as long as such a fusogenic peptide is useful for the affinity separation matrix according to one or more embodiments of the present invention,

The Protein G ligand usable in one or more embodiments of the present invention can be prepared by an ordinary method. Specifically, the DNA encoding the amino acid sequence of the desired Protein G ligand or a fragment thereof is chemically synthesized and amplified by PCR, and the amplified DNA is inserted in a vector. Escherichia coli or the like is infected with the obtained vector and cultivated, and the desired Protein G ligand may be purified from the cultivated bacterial cell or culture medium by chromatography or the like.

The affinity separation matrix used in one or more embodiments of the present invention is prepared by immobilizing the Protein G ligand on an insoluble carrier. The “insoluble carrier” usable in one or more embodiments of the present invention is insoluble in an aqueous solvent which is a solvent of the liquid sample containing an antibody fragment which contains a CH1 region and which does not contain an Fc region, and the insoluble carrier on which the ligand is immobilized can be used for purifying the above-described antibody fragment which specifically binds to the ligand. The insoluble carrier usable in one or more embodiments of the present invention is exemplified by an inorganic carrier such as glass beads and silica gel; an organic carrier; and a composite carrier obtained from the combination of the above carriers, such as an organic-organic composite carrier and an organic-inorganic composite carrier. An organic carrier is exemplified by a carrier composed of a synthetic polymer such as cross-linked polyvinyl alcohol, cross-linked polyacrylate, cross-linked polyacrylamide and cross-linked polystyrene; and a polysaccharide such as crystalline cellulose, cross-linked cellulose, cross-linked agarose and cross-linked dextran. The commercial product thereof is exemplified by porous cellulose gel GCL2000, Sephacryl S-1000 prepared by crosslinking allyl dextran and methylene bisacrylamide through a covalent bond, an acrylate carrier Toyopearl, a cross-linked agarose carrier Sepharose CL4B, and a cross-linked cellulose carrier Cellufine. However, it should be noted that the insoluble carrier usable in one or more embodiments of the present invention is not restricted to the carriers exemplified as the above.

In one or more embodiments, it is preferred that the insoluble carrier usable in one or more embodiments of the present invention has large surface area and that the carrier is porous with a large number of fine pores having a suitable size in terms of a purpose of and method for using the affinity separation matrix according to one or more embodiments of the present invention. The carrier can have any form such as beads, monolith, fiber, and film including hollow fiber, and any form can be selected.

As a method for immobilizing the Protein G ligand as the ligand in one or more embodiments of the present invention on an insoluble carrier, an ordinary method may be applied. For example, the ligand is immobilized by using a reactive group on the surface of an insoluble carrier. Specifically, there is a reactive group such as an amino group, a hydroxy group and a carboxy group on the surface of a general insoluble carrier. The reactive group may be activated or substituted by other reactive group, or a linker group having a reactive group may be introduced on the reactive group. For example, when an epoxy group is introduced on the surface of an insoluble carrier by using epichlorohydrin, diglycidyl ether, 1,4-bis(2,3-epoxypropoxy)butane or the like, or when an iodoacetyl group, a bromoacetyl group, a maleimide group, an N-hydroxysuccinimide group or the like is introduced on the surface of an insoluble carrier, a coupling reaction between the Protein G ligand and the reactive group can be easily accelerated.

When a linker group is used for immobilizing the ligand on an insoluble carrier, the linker group is not particularly restricted. The linker group is exemplified by a C₁₋₆ alkylene group, an amino group (—NH—), an imino group (>C═N— or —N═C<), an ether group (—O—), a thioether group (—S—), a carbonyl group (—C(═O)—), a thionyl group (—C(═S)—), an ester group (—C(═O)— or —O—C(═O)—), an amide group (—C(═O)—NH— or —NH— C(═O)—), a sulfoxide group (—S(═O)—), a sulfonyl group (—S(═O)₂—), a sulfonylamide group (—NH—S(═O)₂— and —S(═O)₂—NH—), and a group formed by binding a plurality of the above-described groups. When the linker group is formed by binding a plurality of the above-described groups, the number of the bound groups may be preferably not more than 10 or not more than 5, and more preferably 3 or less.

A spacer molecule composed of a plurality of atoms may be introduced between the ligand and carrier. Alternatively, the ligand may be directly immobilized on the carrier. In addition, the Protein G ligand according to one or more embodiments of the present invention may be chemically modified for immobilization.

In Step 2, the above-described liquid sample is contacted with the above-described affinity separation matrix in order to selectively bind the antibody fragment on the Protein G ligand in the affinity separation matrix. The specific condition for the contact is riot particularly restricted, and the above-described liquid sample and the above-described affinity separation matrix may be simply mixed. Alternatively, for example, in terms of convenience, it may be preferred that a column is filled with the affinity separation matrix according to one or more embodiments of the present invention to be an affinity column and the liquid sample is flown through the affinity column to selectively adsorb the above-described antibody fragment on the Protein G ligand.

The condition of Step 2 may be appropriately adjusted as long as the above-described antibody fragment contained in the above-described liquid sample is sufficiently adsorbed on the above-described affinity separation matrix. For example, the pH in Step 2 may be adjusted to 6 or more and 8 or less.

Step 3: Step of Washing Affinity Separation Matrix

In Step 3, the affinity separation matrix on which the antibody fragment is adsorbed in the above-described Step 2 is washed to remove an impurity except for the antibody fragment. Even after Step 3, the antibody fragment is adsorbed on the affinity separation matrix. On the one hand, even if an impurity such as a light chain monomer is once adsorbed, the impurity can be removed by Step 3.

As a washing liquid usable for washing the affinity separation matrix in Step 3, a washing liquid which does not disturb the interaction between the antibody fragment and the Protein G ligand is used. For example, water and a buffer of which pH is 5 or more and 8 or less can be used as the washing liquid. A usage amount of the washing liquid is represented in column volume (CV). A column volume is based on an amount of the affinity separation matrix with which a column is filled. For example, when a column is filled with 1 mL-gel of the affinity separation matrix, a usage amount of 1 CV corresponds to 1 mL. A volume of the affinity separation matrix as a standard means a volume of the affinity separation matrix in a gel condition determined by tapping or leaving still the affinity separation matrix in a suspension condition until the volume thereof is not decreased any more. An amount of the washing liquid may be appropriately determined so that an impurity can be sufficiently removed from the affinity separation matrix and may be preferably 3 CV or more, more preferably 4 CV or more, and even more preferably 5 CV or more. When a usage amount of the washing liquid is larger, a removal efficiency of an impurity may become higher and a purity of the antibody fragment in the next step tends to be higher but a recovery yield may be decreased. Nevertheless, when the affinity separation matrix having a SpG variant or a SpG domain variant of which binding ability to a CH1 region is high as a Ligand is used, a high recovery yield may be achieved even in the case where a usage amount of the washing liquid is large. For example, when a chromatography system is used, it can be judged without difficulty whether an impurity can be sufficiently removed or not by monitoring an elution profile.

An impurity is exemplified by an impurity derived from an antibody in addition to a protein and DNA derived from a cultivated host cell. An impurity derived from an antibody is exemplified by a substance derived from an excessively produced antibody and a component generated by misfolding, such as a light chain monomer, a light chain dimer, an antibody aggregate. Since an impurity derived from an antibody has a physical property similar to that of a target antibody fragment, it is generally difficult to separate the impurity. On the one hand, such an impurity derived from an antibody can be efficiently removed by one or more embodiments of the present invention method. The above-described impurity can be detected by SDS-PAGE, HPLC or the like, but the detection method is not restricted thereto.

Step 4: Step of Separating Antibody Fragment

In Step 4, the above-described antibody fragment is separated from the affinity separation matrix on which the antibody fragment is adsorbed by using an eluate. By Step 4, the purified antibody fragment can be obtained.

In one or more embodiments of the present invention, as an eluate to elute the antibody fragment, an acidic aqueous solution can be used. Such an acidic aqueous solution is exemplified by aqueous solutions of acetic acid, citric acid and glycine. When the pH of the aqueous solution is low, the antibody fragment can be efficiently eluted and an amount of the eluate can be reduced. When the pH of the aqueous solution is high, it becomes possible to reduce the damage caused by acid to the antibody fragment. In one or more embodiments, the pH of the eluate is preferably 2.5 or more and 4.0 or less. When the pH is 2.5 or more, the chemical change or the like of the antibody fragment can be suppressed more surely. On the one hand, when the pH is 4.0 or less, the antibody fragment can be eluted more surely. In one or more embodiments, the pH is more preferably 2.8 or more, even more preferably 3.0 or more, and more preferably 3.8 or less, even more preferably 3.5 or less. The purity of the obtained antibody fragment can be determined by SDS-PAGE, HPLC or the like, but the method is not restricted thereto.

Step 5: Posttreatment Step

By the above-described Step 4, an aqueous solution of the antibody fragment is obtained. The antibody fragment may be further purified by salting-out, chromatography, recrystallization or the like, and dried by freeze dry, spray dry, film drying method or the like.

Step 6: Step of Regenerating Affinity Separation Matrix

In Step 6, the affinity separation matrix which is used in the above-described Step 4 and from which the antibody fragment is separated is regenerated by washing with a regenerating solution. It is not needed to necessarily perform Step 6 after the above-described Step 4, and Step 6 may be performed once every three iterations of the above Steps 1 to 3, once every five iterations, or once every ten iterations. Specifically, when a performance of the affinity separation matrix, such as binding capability, is maintained, Step 6 is not necessarily performed. The implementation frequency and condition of Step 6 is also different depending on the liquid sample containing the antibody fragment to be purified.

The “regenerating solution” usable for the regeneration of the affinity separation matrix means an aqueous solution by which purpose such as washing and sterilization can be achieved. More specifically, 1 M acetic acid solution (pH 2.0), 20 mM phosphate-1% SDS solution (pH 7.0), 6 M guanidine-hydrochloride solution (pH 7.0), 70% ethanol, 0.1 M hydrochloric acid (pH 1.0), 8 M urea solution (pH 10.5), 0.1 M glycine-sodium hydroxide solution (pH 11) can be used as a regenerating solution, but the regenerating solution is not restricted thereto.

The time to treat the affinity separation matrix after the above-described Step 4 by the regenerating solution is not particularly restricted and may be appropriately adjusted, since a damage degree of the Protein G ligand is different depending on the kind of the regenerating solution and the temperature at the treatment. For example, when 0.1 M hydrochloric acid (pH 1.0) is used as a regenerating solution and the temperature during immersion is atmospheric temperature, the time may be preferably 1 hour and more preferably 2 hours. When 8 N urea. (pH 10.5) is used as a regenerating solution and the temperature during immersion is atmospheric temperature, the time may be preferably 1 hour and more preferably 2 hours.

The affinity separation matrix regenerated by Step 6 can be used in the above-described Steps 1 to 3 again.

The present application claims the benefit of the priority date of Japanese patent application No. 2016-148820 filed on. Jul. 28, 2016. All of the contents of the Japanese patent application No. 2016-148820 filed on July 28, 2016, are incorporated by reference herein.

### EXAMPLES

Hereinafter, one or more embodiments of the present invention are described in more detail with Examples; however, the present invention is not restricted to the following Examples.

### Example 1

(1) Preparation of Fab Fragment-Containing Supernatant

A Fab fragment was designed on the basis of the public information of the sequence of completely humanized anti-TNF α antibody (adalimumab) and selected as an antibody fragment which contained a heavy chain constant region (CH region) but which did not contain an Fc region. A Gene encoding the Fd chain amino acid sequence (CH1 region and VH region, SEQ ID NO: 3) and the light chain amino acid sequence (SEQ ID NO: 4) of the above-described anti-TNF-α antibody was designed and chemically synthesized. The Fab gene was prepared by PCR using the gene as a template. The above-described Fab fragment was produced by using the obtained gene and methylotrophic yeast. The yeast used for producing the Fab fragment was obtained and cultivated in accordance with the method of Examples 1, 8 and 9 of WO 2012/102171. The culture medium containing the produced Fab fragment was centrifuged and the supernatant was recovered. The recovered supernatant was filtrated by using a sterilized filter having a pore diameter of 0.22 μm (“Minisart” manufactured by Sartorius).

(2) Preparation of Protein G variant carrier

A carrier on which the Protein G domain variant described in WO 2016/031902 was immobilized was prepared. Specifically, a Fab region-binding peptide was immobilized on a cellulose carrier. The Fab region-binding peptide had a 2 domains structure composed of the amino acid sequence of SEQ ID NO: 5 added by the linker sequence between domains and the C-terminal sequence of wild Protein C and Cys at the C-terminal. As the cellulose carrier, crystalline highly-crosslinked cellulose (manufactured by JNC Corporation, corresponding to the gel obtained by the method described in JP 2009-242770 A) was used. The association constants of the above-described Fab region-binding peptide to the Fab of anti-EGFR antibody and anti-TNF α were 10⁶ M⁻¹or more. The immobilization was performed by similar method to the epoxy-immobilization method described in WO 2016/031902.

(3) Purification of Fab from Fab Fragment-Containing Supernatant

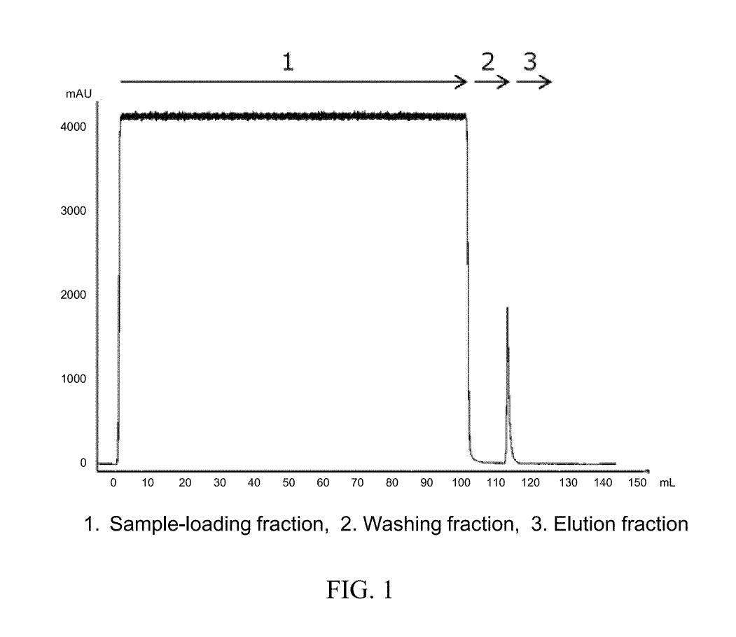

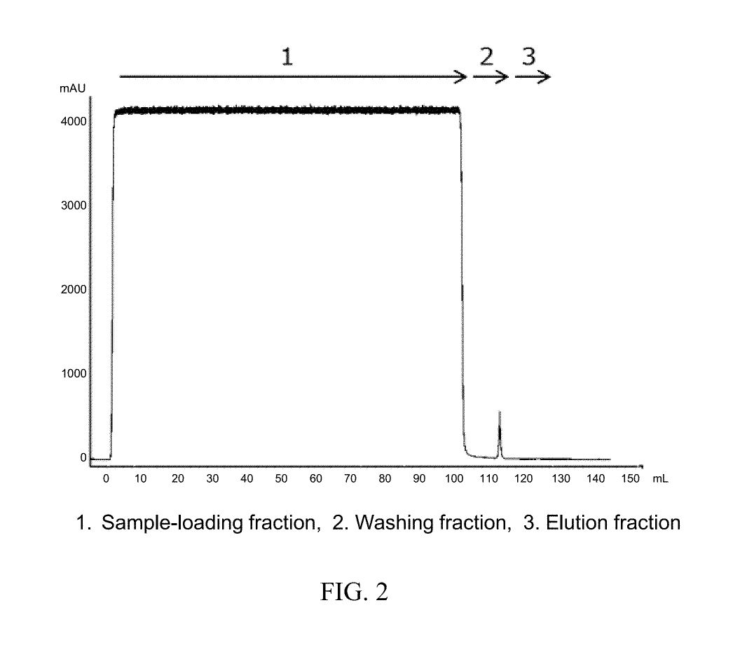

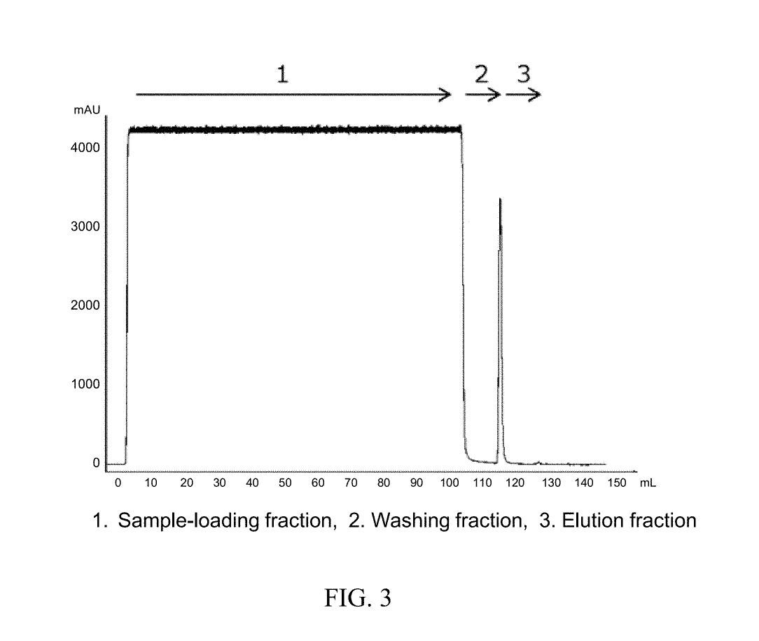

In order to adjust the pH of the Fab fragment-containing supernatant prepared in the above-described (1), the supernatant was diluted 2-fold by using an equilibrating buffer (20 mM NaH₂PO₄—Na₂HPO₄, 150 mM NaCl, pH 7.4). Then, after the supernatant was filtrated by using a filter having a pore diameter of 0.22 μm (“Minisart” manufactured by Sartorius), the Fab fragment was purified by using a commercially available Protein G carrier (“Protein G Sepharose 4 Fast Flow” manufactured by GE Healthcare) or the Protein G variant carrier prepared in the above-described (2). For comparison, the Fab fragment was also purified by using a commercially available Protein L carrier (“Capto L” manufactured by GE Healthcare). A commercially available column (“Tricorn 5/50” manufactured by GE Healthcare) was filled with 1 mL-gel of the carriers and connected to chromatography system AKTAavant 25 (manufactured by GE Healthcare). Specifically, the following procedure was performed. First, 5 CV (column volume) of an equilibrating buffer (20 mM Na₂HPO₄—NaH₂PO₄, 150 mM NaCl, pH 7.4) was flown through the column to equilibrate the carrier. Then, the Fab fragment-containing supernatant prepared in the above-described (1) was diluted 2-fold with the equilibrating buffer to be a sample, and 100 mL of the sample was supplied to the column. Next, after the carrier was washed by flowing 10 CV of the equilibrating buffer, 10 CV of an elution liquid (100 mM glycine-HCl, pH 2.7) was flown to elute the adsorbed Fab fragment. Then, after 3 CV of the equilibrating buffer was flown and 5 CV of 1 M acetic acid aqueous solution was flown to wash the carrier, 5 CV of the equilibrating buffer was flown to complete the purification. The flow rate was adjusted to 1 mL/min in the above procedure. In the any purifications using the carriers, the fractions at the time of the supply of the sample, washing and elution were kept. The obtained elution fraction was neutralized by using 2 M Tris solution. Each chromatogram in the case where a Protein G variant carrier, a commercially available Protein G carrier and a commercially available Protein L carrier were used is shown as FIGS. 1 to 3. In FIGS. 1 to 3, each fraction was represented as 1 to 3.

(4) Identification of Component in Each Fraction

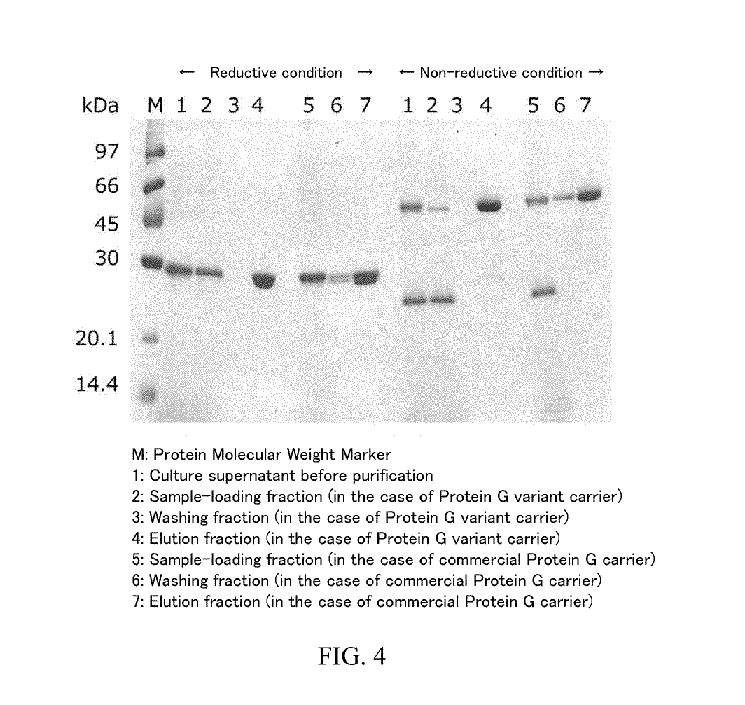

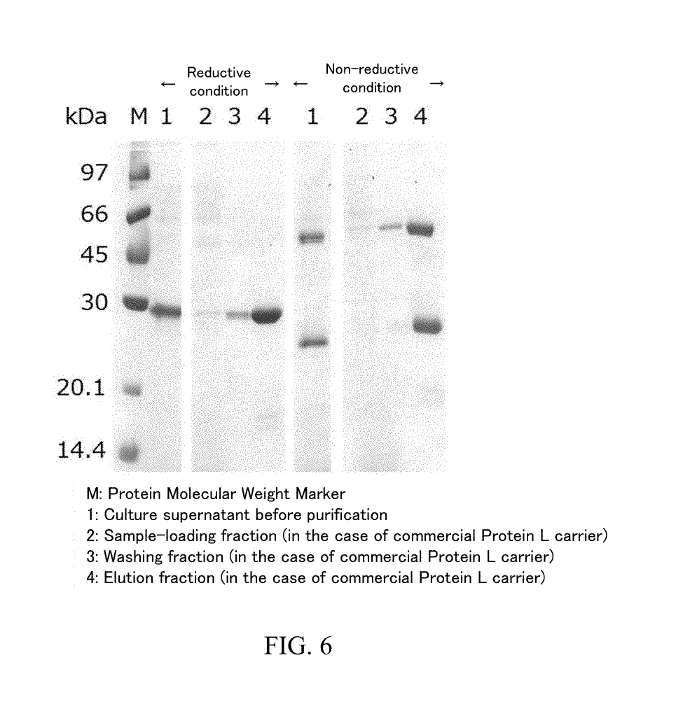

The sample loading fractions, washing fractions and elution fractions obtained by using each carrier in the above-described (3) were analyzed by SDS-PAGF. Specifically, SDS-PAGE was performed by using mini-slab electrophoresis system with a built-in power “PageRun” manufactured by ATTO Corporation and 15% polyacrylamide-precast gel “e.PAGEL” manufactured by ATTO Corporation in accordance with the attached manual. The sample loading fraction was diluted by 10-fold, since the protein concentration was high. The gel was stained and decolorized by using CBB staining solution used for visualization of a protein (“EzStain AQua” manufactured by ATTO Corporation) in accordance with the attached manual. The SDS-PAGE result in the case where a Protein G variant carrier and a commercially available Protein G carrier were used is shown as FIG. 4, the enlarged photograph in the case of non-reductive treatment condition is shown as FIG. 5, and the SDS-PAGE result in the case where a commercially available Protein IL carrier was used is shown as FIG. 6

As the result demonstrated by FIG. 4, the band positions of the light chain (molecular weight: 23,412) and the heavy chain (VH region and CH1 region: 23,871) were slightly different but observed around 30 kDa in a reductive condition. In a non-reductive condition, the band of the Fab fragment was observed between 45 kDa and 66 kDa, and a band was also observed between 20.1 kDa and 30 kDa. As the SDS-PAGE result under non-reductive condition demonstrated by FIG. 6, when a Protein L carrier was used, the band between 20.1 kDa and 30 kDa was not observed in the sample loading fraction but was observed in the washing fraction. Since a Protein L carrier has a binding ability to a κ light chain, the band can be identified as a component derived from a light chain and is considered to be a light chain monomer from the aspect of the molecular weight. The position of the light chain monomer band in a reductive condition was different from that in a non-reductive condition. Since there is cysteine in the light chain amino acid sequence, the structure of the light chain in a reductive condition is different from that in a non-reductive condition. The difference of the positions is considered to be due to the difference of the structures.

According to FIG. 5 which is an expanded figure around 45 to 66 kDa of FIG. 4 in a non-reductive condition, there are 2 bands between 45 kDa and 66 kDa in the culture supernatant before the purification. The band having lower molecular weight is considered to be the band of the light chain dimer.

As the result demonstrated in FIG. 6, when a Protein L carrier was used, both of the light chain monomer and light chain dimer were mixed in the elution fraction and could not be separated from the Fab fragment.

On the one hand, as FIG. 4, when any of a Protein G variant carrier and a commercially available Protein G carrier were used, the band of the light chain monomer existed in the culture supernatant before the purification and the sample loading fraction but could not be observed in the washing fraction and elution fraction. In addition, the band of the light chain dimer was observed in both of the sample loading fractions in the cases of a Protein G variant carrier and a commercially available Protein G carrier but could not observed in the washing fraction and elution fraction. From the results, it was found that an unwanted component and a misfolded protein such as a light chain monomer and a light chain dimer in a culture supernatant can be removed and a Fab fragment can be purified with high purity by using a Protein G carrier.

When a commercially available protein G carrier was used, the fact that there was the band of the Fab in both of the sample loading fraction and washing fraction was different from the case where a protein G variant carrier was used. In other words, the result suggests that a light chain monomer and a light chain dinner can be removed by a commercially available protein G carrier but a Fab fragment was leaked at the time of a sample loading and washing. On the one hand, when a Protein G variant carrier was used, the Fab fragment was not contained in the washing liquid. Thus, the carrier on which a Protein G variant having high association constant to a Fab fragment is immobilized has more excellent ability to maintain a Fab fragment. As a result, a Fab fragment having high purity can be obtained with high recovery yield by the purification step using the carrier.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A method for producing an antibody fragment, comprising:
preparing a liquid sample that comprises the antibody fragment and does not comprise an Fc fragment;
adsorbing the antibody fragment on an affinity separation matrix by contacting the liquid sample with the affinity separation matrix;
removing impurities from the affinity separation matrix by washing the affinity separation matrix; and
separating the antibody fragment from the affinity separation matrix,
wherein the antibody fragment comprises a CH1 region and does not comprise an Fc region,
wherein the affinity separation matrix comprises a water-insoluble carrier and a ligand immobilized on the water-insoluble carrier, and
wherein the ligand is Protein G, a Protein G domain, a Protein G variant or a Protein G domain variant.

2. The method according to claim 1, wherein the ligand is the Protein G variant or the Protein G domain variant, and
wherein an association constant of the Protein G variant or the Protein G domain variant to the CH1 region is 10⁶ M⁻¹ or more.

3. The method according to claim 2, wherein the ligand is the Protein G variant or the Protein G domain variant, and
wherein the Protein G variant or the Protein G domain variant has an amino acid sequence of SEQ ID NO: 5.

4. The method according to claim 1, wherein the antibody fragment comprises a light chain, and
wherein the impurities comprises one or more selected from the group consisting of a light chain monomer, a light chain dimer and an antibody aggregate.

5. The method according to claim 1, wherein removing the impurities is performed by washing the affinity separation matrix using 3 column volume or more of a washing liquid.

6. The method according to claim 1, wherein separating the antibody fragment from the affinity separation matrix is performed by adding an eluate to the affinity separation matrix, and
wherein the eluate is an aqueous solution comprising one or more acids selected from the group consisting of acetic acid, citric acid and glycine.

7. The method according to claim 6, wherein a pH of the aqueous solution is 2.5 to 4.0.


SEQUENCE LISTING (5 sequences)

SEQ ID NO: 1 - protein, 56 aa, Streptococcus sp. GX7809
DTYKLILNGKTLKGETTTEAVDAATAEKVFKQYANDNGVDGEWTYDDATKTFTVTE

SEQ ID NO: 2 - protein, 56 aa, Streptococcus sp. GX7809
TTYKLVINGKTLKGETTTKAVDAETAEKAFKQYANDNGVDGVWTYDDATKTFTVTE

SEQ ID NO: 3 - protein, 224 aa, Homo sapiens
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 4 - protein, 214 aa, Homo sapiens
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 56 aa, Artificial Sequence
  SpG domain mutant
TTYKLILNGKTLTGYTTAIAADAATAEIVLKQFANDNGIDGEWTYDDATKTFTVTE